(19) United States
(12) Patent Application Publication
(10) Pub. No.: US 2007/0092552 A1
(43) Pub. Date: Apr. 26, 2007

(54) Chewable lozenge cold remedy composition and method for making same

(76) Inventor: Tim Clarot, Phoenix, AZ (US)

Correspondence Address: Cynthia L Pillote;Snell & Wilmer, One Arizona Center, 400 East Van Buren, Phoenix, AZ, 85004-2202, US

(21) Appl. No.: 10/556,739

(22) PCT Filed: Apr. 30, 2004
(86) PCT No.: PCT/US04/13421

§ 371 Date: Nov. 13, 2006

Related U.S. Application Data
(60) Provisional application No. 60/467,010, filed on Apr. 30, 2003.

(51) Int. Cl. A61K 31/315; A61K 9/68

(52) U.S. Cl. 424/440; 514/494

(57) ABSTRACT

A chewable lozenge for the treatment of cold symptoms having a zinc formulation which includes both zinc gluconate and zinc actetate.

### FIELD OF INVENTION

The present invention generally relates to a composition for reducing cold symptoms and their duration. More particularly, the present invention relates to a chewable lozenge having one or more active substances including zinc gluconate and/or zinc acetate for treating cold symptoms and to a method for making the chewable lozenge.

### BACKGROUND OF THE INVENTION

The common cold is a widespread malady having a number of irritable and uncomfortable symptoms such as headaches, muscle aches and pains, fever, congested or runny nose, sore throat and watery eyes. Bacteria and viruses which cause colds are usually present in the mouth, throat, pharynx, and nasal passages. Therefore, colds are typically treated with oral compounds or compounds delivered into the nasal passages.

Zinc is a known active substance for controlling the bacteria and viruses that cause common colds and the oral and topical administration of zinc and zinc containing compounds have long been utilized in the treatment and prophylaxis of the common cold. For example, a number of patents have been issued to George A. Eby, III, which disclose zinc containing compounds for treating the symptoms of, or curing, the common cold.

Eby's U.S. Pat. No. 4,503,070 issued in 1995 discloses the use of a hard lozenge containing zinc gluconate to reduce the duration of a cold and his U.S. Pat. No. 5,409,905 discloses a composition having a highly ionizable zinc compound which provides sustained release of Zn²⁺ ions. The highly ionozable zinc compound is selected from zinc acetate, zinc propionate, zinc butyrate, zinc betahydroxybutyrate, zinc benzoate, zinc formate, and mixtures thereof while the composition specifically excludes flavor masking amounts of anethole and strong zinc chelators.

U.S. Pat. No. 4,956,385 issued to Eby, III describes the method of applying an ionizable zinc compound other than zinc gluconate to the oral mucosa for treating the common cold. Later, in 1990, Eby's U.S. Pat. No. 4,503,070 was reissued as Re. 33,465 and included claims similar to Eby's '385 patent with the exception that the ionizable zinc compound was specifically identified and defined as being zinc gluconate.

Finally, U.S. Pat. Nos. 5,002,970 and 5,095,035 were issued to Eby, III in 1999 and 1992, respectively, which disclosed oral compositions for releasing zinc ions which included an anethole in an amount to flavor-mask the zinc aftertaste, or a sweet pharmaceutically acceptable carrier. Zinc gluconate, zinc acetate and zinc ascorbate were all identified as individual possibilities for the ionizable zinc compound used with an anethole while zinc acetate was identified as an ionizable zinc compound for use with a sweet pharmaceutically acceptable carrier.

Later, in 1997, two patents were issued to Bryce-Smith, namely U.S. Pat. No. 5,622,724 and U.S. Pat. No. 5,688,531, which disclosed nasal, oral and opthalmological sprays having a dilute solution of unchelated zinc ion for treating cold and allergy symptoms. Zinc sulfate and/or zinc chloride were specifically identified as acceptable selections for the unchelated zinc ion.

Still later, U.S. Pat. No. 6,139,864 was issued to Durr et al. in 2000. The Durr et al. patent describes foods and pharmaceuticals containing zinc and an antimicrobially effective amount of a sugar alcohol mixture. The zinc was identified as being present in the form of zinc gluconate or zinc acetate.

Although the previously described patents disclose the use of various forms of zinc, including zinc gluconate or zinc acetate, for oral application in reducing and treating the occurrence of colds and their symptoms, none of the prior art references disclose chewable lozenge to provide an effective oral application for reducing and/or preventing colds and their symptoms.

### SUMMARY OF THE INVENTION

The present invention is directed to an oral composition and method for treating symptoms associated with the common cold. In accordance with various aspects of the present invention, a cold remedy composition is formulated to permit one or more active substances to come in contact with the oral membrane and be adsorbed by the oral, oralpharyngeal, and nasal membranes.

In accordance with various embodiments of the invention, a combination of zinc salts provides a higher concentration of zinc with a quick ionization potential that is capable of being sustained over a more lengthy period of time. The present invention also achieves a balance between efficacy and consumer acceptance by formulating a combination of zinc gluconate and zinc actetate to a palatable stage.

In accordance with one exemplary embodiment of the invention, a chewable lozenge is formulated to include a zinc formulation containing both zinc gluconate and zinc actetate. In accordance with various aspects of the invention, the active substances include homeopathic amounts of their active agents.

The chewable lozenge preferably includes a thickener and emulsifier to create and maintain its form as a chewable lozenge. In accordance with further embodiments, the chewable lozenge may include one or more of a stabilizer, a sweetener, texture additives, flavor enhancers, and coloring.

In accordance with various aspects of the invention, the chewable lozenge has a total weight of approximately 6.5 grams and one chewable lozenge is administered to the mouth about every three hours.

The present invention is also directed to a method for treating cold symptoms utilizing the chewable lozenge as well as a method for making the chewable lozenge of the present invention having both zinc gluconate and zinc actetate in its formulation.

These and other advantages of the various compositions and methods according to various aspects of the present invention will be apparent to those skilled in the art upon reading and understanding the detailed description below.

### DETAILED DESCRIPTION OF EXEMPLARY EMBODIMENTS

The present invention provides improved compositions and methods for treating cold symptoms. The chewable lozenge is consumed while being held in the mouth by chewing, sucking, and/or dissolving.

In accordance with exemplary embodiments of the invention, a chewable lozenge to reduce cold symptoms includes one or more active ingredients. In accordance with various embodiments of the invention, the lozenge includes active ingredients such as zinc gluconate and zinc acetate, and a bulk substance, such as sweeteners and thickeners, which comprises part of the carrier for the active ingredients. As described below, the chewable lozenge may also include additional ingredients such as stabilizers, texture agents, preservatives, emulsifiers, colorings, flavor enhancers, and other taste modifiers.

As used herein, an active substance includes any of one or more substances that produces or promotes a beneficial therapeutic, physiological, homeopathic, allopathic and/or pharmalogical effect on the body. Such beneficial effects may be brought upon any animal or human patient, and various systems associated therewith, including the immune system, respiratory system, circulatory system, nervous system, digestive system, urinary system, endocrine system, muscular system, skeletal system, and the like, as well as any organs, tissues, membranes, cells, and subcellular components associated therewith.

As will be appreciated by those skilled in the art, beneficial effects include assisting the more efficient functioning of the various systems described above, such as, for example, helping the body fight sickness and disease, helping the body to heal, etc. Exemplary active substances include any element, composition or material producing a beneficial effect, including vitamins, minerals, nucleic acids, amino acids, peptides, polypeptides, proteins, genes, mutagens, antiviral agents, antibacterial agents, anti-inflammatory agents, decongestants, histamines, anti-histamines, anti-allergens, allergy-relief substances, homeopathic substances, pharmaceutical substances, and the like.

Exemplary active substances include metallic and ionic zinc, which is thought to bind to I-CAM receptors within the oralphryngeal-nasal cavity to inhibit the spread of the virus. When a composition comprising zinc is applied to the oral cavity, zinc ions from the composition adhere to a portion of the membrane in the oral cavity. It is believed that the zinc in the mucous or mucous membrane creates a barrier which inhibits viral infection of the oral membrane.

In accordance with one embodiment of the invention, a homeopathic concentration of zinc ions in the zinc composition of the invention is about 9.00 mg to about 11.00 mg and preferably about 10.00 mg. Zinc gluconate is preferably present in an amount of about 0.32 weight % to about 0.42 weight %, and most preferably about 0.37 weight %. Zinc actetate is preferably present as zinc acetate dihydrate in an amount of about 0.45 weight % to about 0.55 weight %, and most preferably 0.50 weight %.

Sweeteners comprise the majority of the carrier and include sugar and maltitol. In one exemplary embodiment, confectioners sugar is present in an amount of about 51.82 weight % and maltitol syrup is present in an amount of about 16.00 weight %.

The chewable lozenge may also include food-grade or pharmaceutical grade thickeners such as, for example, carrageenan, sugar, guar gum, aloe vera, cellulose, methylcellulose, and the like. In one exemplary embodiment, the chewable lozenge preferably comprises about 0.5 weight % hydroxypropyl methylcellulose.

The chewable lozenge of the present invention may also include a stabilizer such as glycerin, or the like which function to keep the zinc in its ionic form. In a preferred embodiment, the stabilizer includes glycerin present in an amount of about 3.00 weight % of the chewable lozenge.

The chewable lozenge may also include emulsifiers, texture agents, preservatives, antiseptics, permeation enhancers, sequestering agents, buffers, flavor enhancers, and coloring agents.

Exemplary texturing agents include maltodextrin, monoglycerides, diglycerides, cottonseed oil, and soybean oil. The present invention may include any combination of these and/or other texturing agents. In one exemplary embodiment, the chewable lozenge includes maltodextrin in an amount of about 15.00 weight %, mono and diglycerides in an amount of about 2.50 weight %, and partially hydrogenated cottonseed/soybean oil in an amount of about 5.00 weight %.

Lecithin functions as an exemplary emulsifier in the present invention. In one exemplary embodiment, a chewable lozenge includes lecithin in an amount of about 0.77 weight %.

In accordance with various exemplary embodiments of the invention, a chewable lozenge may include a flavor enhancer such as, for example, spray dried strawberry flavor in an amount of about 2.50 weight % and/or a coloring agent such as, for example, FD&C Red #40 in an amount of about 0.04 weight %.

In accordance with another aspect of the invention, a preservative may be added to the composition to facilitate stability of the various ingredients. Any suitable preservative may be used in accordance with the present invention. Suitable exemplary preservatives for use with the present invention may include benzalkonium chloride and disodium EDTA.

As noted above, the composition may also include permeation enhancers, which are believed to function by enlarging or loosening tight junctions between cells in the oral membrane, thereby facilitating passage of the active substance therethrough. Exemplary permeation enhancers include liposomes, sequestering agents, ascorbic acid (Vitamin C), glycerol, chitosan, and lysophosphotidylcholin, or any other substance that provides a similar function or result. By way of example, the permeation enhancer may include a sequestering agent, such as EDTA. EDTA is thought to chelate calcium. When applied to the nasal membrane, it is believed to remove calcium from the cell junctions, thereby loosening the junctions to facilitate passage of an active substance therethrough.

Permeation enhancers may be present in any effective amount, with preferably concentrations ranging from about 0.00001% to about 5.0% by weight. In a preferred embodiment, the permeation enhancer includes disodium EDTA, at a concentration of about 0.0001% to about 1.0% by weight, and preferably at about 0.10% by weight.

The chewable lozenge of the present invention is delivered to the oral cavity and then masticated within the oral cavity until completely dissolved and swallowed. The chewable lozenge preferably has a total weight of about 6.5 grams and is administered in a dosage of about one chewable lozenge every 3 hours.

The Examples set forth below are illustrative of various aspects of certain preferred embodiments of the present invention. The compositions, methods and various parameters reflected therein are intended only to exemplify various aspects and embodiments of the invention, and are not intended to limit the scope of the claimed invention.

### EXAMPLE 1

An exemplary chewable lozenge for delivering an active zinc substance to the oral cavity is prepared by admixing and forming the following ingredients:

| | | | | Quantity |
|---|---|---|---|---|
| Generic Name | Brand Name | Grade | Manufacturer | (wt %) |
| Zinc Acetate | | USP/ACS | American | 0.5 |
| Dihydrate | | | International | |
| | | | Chemical Inc. | |
| | | | (AIC) | |
| Zinc Gluconate | | USP/NF | American | 0.37 |
| | | | International | |
| | | | Chemical Inc. | |
| | | | (AIC) | |
| Maltitol Syrup | Lycasin HBC | GRAS | Roquette | 16.00 |
| Sugar 10× | Confectioners | GRAS | Domino | 51.82 |
| | Sugar | | | |
| Maltodextrin | Fibersol-2 | GRAS | Matsutani | 15.00 |
| Part. | Astral s | GRAS | ACH | 5.00 |
| Hydrogenated | flakes | | | |
| Cottonseed/soy | | | | |
| oil | | | | |
| Glycerin | superol | USP | Proctor and | 3.00 |
| | | | Gamble | |
| Mono and | Panalile 90DK | GRAS | ADM Arkady | 2.50 |
| Diglycerides | | | | |
| Hydroxypropyl | Methocel K 99 | USP | Dow | 0.5 |
| Methycellulose | | | | |
| Lecithin | Yelkin T | GRAS | ADM | 0.77 |
| Strawberry | | | David Michael | 2.5 |
| flavor Spray | | | & Co. | |
| dried | | | | |
| FD&C red #40 | Red #40 dye | FD&C | Sensient | 0.04 |

The above ingredients were formed into a chewable lozenge in accordance with standard manufacturing techniques known in the art for forming chewable lozenges.

### EXAMPLE 2

An exemplary chewable lozenge for delivering an active zinc substance to the oral cavity is prepared by admixing and forming the following ingredients:

| | | | | Quantity |
|---|---|---|---|---|
| Generic Name | Brand Name | Grade | Manufacturer | (wt %) |
| Zinc Gluconate | | USP/NF | American | 0.42 |
| | | | International | |
| | | | Chemical Inc. | |
| | | | (AIC) | |
| Maltitol Syrup | Lycasin HBC | GRAS | Roquette | 16.00 |
| Sugar 10× | Confectioners | GRAS | Domino | 51.82 |
| | Sugar | | | |
| Maltodextrin | Fibersol-2 | GRAS | Matsutani | 15.00 |
| Part. | Astral s | GRAS | ACH | 5.00 |
| Hydrogenated | flakes | | | |
| Cottonseed/soy | | | | |
| oil | | | | |
| Glycerin | superol | USP | Proctor and | 3.00 |
| | | | Gamble | |
| Mono and | Panalile 90DK | GRAS | ADM Arkady | 2.50 |
| Diglycerides | | | | |
| Hydroxypropyl | Methocel K 99 | USP | Dow | 0.5 |
| Methycellulose | | | | |
| Lecithin | Yelkin T | GRAS | ADM | 0.77 |
| Strawberry | | | David Michael | 2.5 |
| flavor Spray | | | & Co. | |
| dried | | | | |
| FD&C red #40 | Red #40 dye | FD&C | Sensient | 0.04 |

In accordance with Example 1 above, a chewable lozenge for reducing the severity and duration of symptoms of the common cold has been presented. The chewable lozenge is delivered to the oral cavity and then masticated until completely dissolved and swallowed. The zinc formulation included in the chewable lozenge contains zinc gluconate and zinc acetate in order to increase the zinc concentration and the ionization potential of zinc, and to sustain the availability of ionizable zinc for adsorption into the oral, oralpharyngeal, and nasal membranes.

The present invention has been described above with reference to exemplary embodiments and examples. It should be appreciated that the particular embodiments shown and described herein are illustrative of the invention and its best mode and are not intended to limit in any way the scope of the invention as set forth in the claims. Those skilled in the art having read this disclosure will recognize changes and modifications may be made to the exemplary embodiments without departing from the scope of the present invention. For example, artisans will recognize that reference to oral, oralpharyngeal, and nasal membranes includes any interior surface of the oral and nasal cavities permitting delivery of an active substance, such as the zinc formulation, to the body, including the epithelial layer of the membranes or mucous of the epithelial layer of the membranes. Further, though reference is made both to “substances” and “ingredients”, skilled artisans will further appreciate that the two terms can be used interchangeably.

Additionally, although certain components were described herein as being included in the oral composition in addition to the zinc formulation and the pharmaceutically acceptable carrier, it should be understood that carrier may be referred to as also including those certain components and that any suitable carrier may be achieved through any number of combination of additives now known or hereinafter devised. Accordingly, these and other changes or modifications are intended to be included to be within the scope of the present invention, as expressed in the following claims.

I claim:

1. A chewable lozenge for delivering an active substance to at least one of an oral membrane, an oral pharyngeal membrane and a nasal membrane by administration through the oral cavity, said chewable lozenge comprising:
a pharmaceutically acceptable carrier; and
a zinc formulation comprising both zinc gluconate and zinc actetate.

2. The chewable lozenge of claim 1, wherein said chewable lozenge comprises zinc gluconate in an amount of about 16 to 19 mg and zinc acetate in an amount of about 24 to 26 mg.

3. The chewable lozenge of claim 2, wherein said chewable lozenge preferably comprises zinc gluconate in an amount of about 17.42 mg and zinc acetate in an amount of about 25.16 mg.

4. The chewable lozenge of claim 1, wherein said chewable lozenge comprises free ionic zinc in an amount of about 9 to 11 mg.

5. The chewable lozenge of claim 4, wherein said chewable lozenge preferably comprises free ionic zinc in an amount of about 10.00 mg.

6. The chewable lozenge of claim 1, wherein said pharmaceutically acceptable carrier comprises about 50 to 90 weight % of said chewable lozenge.

7. The chewable lozenge of claim 6, wherein said pharmaceutically acceptable carrier comprises at least one of maltitol, maltodextran, and sugar.

8. The chewable lozenge of claim 1, wherein said zinc formulation comprises about 0.5 to 2.0 weight % of said chewable lozenge.

9. The chewable lozenge of claim 1 further comprising a stabilizer.

10. The chewable lozenge of claim 9, wherein said stabilizer comprises glycerin.

11. The chewable lozenge of claim 10, wherein said stabilizer preferably comprises glycerin in an amount of about 3.0 weight %.

12. The chewable lozenge of claim 1 further comprising a sweetener.

13. The chewable lozenge of claim 12, wherein said sweetener comprises at least one of the following: maltitol, fructose, and sucrose.

14. The chewable lozenge of claim 13 wherein said sweetener preferably comprises sucrose in an amount of about 52 weight %.

15. The chewable lozenge of claim 1 further comprising a preservative.

16. The chewable lozenge of claim 1 further comprising a flavor enhancer.

17. The chewable lozenge of claim 16, wherein said flavor enhancer preferably comprises about 2.5 weight % of said chewable lozenge.

18. The chewable lozenge of claim 1 further comprising an emulsifier.

19. The chewable lozenge of claim 18, wherein said emulsifier preferably comprises about 0.77 weight % lecithin.

20. The chewable lozenge of claim 1 further comprising at least one texture agent.

21. The chewable lozenge of claim 20, wherein said texture agent comprises at least one of the following: maltodextrin, monoglycerides, diglycerides, cottonseed oil, soybean oil, and droxypropyl methyl-cellulose.

22. The chewable lozenge of claim 21, wherein said texture agent comprises about 15.00 weight % maltodextrin, about 2.50 weight % mono and diglycerides, and about 5.00 weight % cottonseed/soybean oil.

23. The chewable lozenge of claim 1 further comprising a coloring agent.

24. A chewable lozenge for delivering an active substance to at least one of an oral, an oral pharyngeal, and a nasal membrane by administration through the mouth, said chewable lozenge comprising:
about 86 to about 99.9999 weight % of a pharmaceutically acceptable carrier;
about 0.2 to about 1.0 weight % of zinc gluconate; and
about 0.2 to about 1.0 weight % of zinc acetate.

25. The chewable lozenge of claim 24, wherein said chewable lozenge comprises free ionic zinc in an amount of about 9 to 11 mg.

26. The chewable lozenge of claim 25, wherein said chewable lozenge preferably comprises free ionic zinc in an amount of about 10.00 mg/mi.

27. The chewable lozenge of claim 24, wherein said zinc formulation comprises about 0.4 to 2.0 weight % of said chewable lozenge.

28. A method for delivering an active substance to at least one of an oral, an oral pharyngeal, and a nasal membrane by administration through the mouth, the method comprising the steps of:
providing a chewable lozenge having a pharmaceutically acceptable carrier and a zinc formulation comprising both zinc gluconate and zinc acetate;
inserting the chewable lozenge into the oral cavity; and
masticating the chewable lozenge until completely dissolved and swallowed.

29. The method of claim 28 wherein zinc gluconate is present in an amount of about 16 to 19 mg and zinc acetate is present in an amount of about 24 to 26 mg.
